(19) United States
(12) Plant Patent Application Publication
(10) Pub. No.: US 2002/0100101 P1
(43) Pub. Date: Jul. 25, 2002

(54) DAHLIA PLANT NAMED 'DIANA, PRINCESS OF WALES'

(76) Inventor: ROBIN MARKS, AYLESBURY (GB)

Correspondence Address: CHRISTIE PARKER & HALE, P O BOX 7068, PASADENA, CA, 911097068

(21) Appl. No.: 09/250,446

(22) Filed: Feb. 16, 1999

(51) Int. Cl. A01H 5/00

(52) U.S. Cl. PLT/321

(57) ABSTRACT

A dahlia plant variety producing pink flowers.

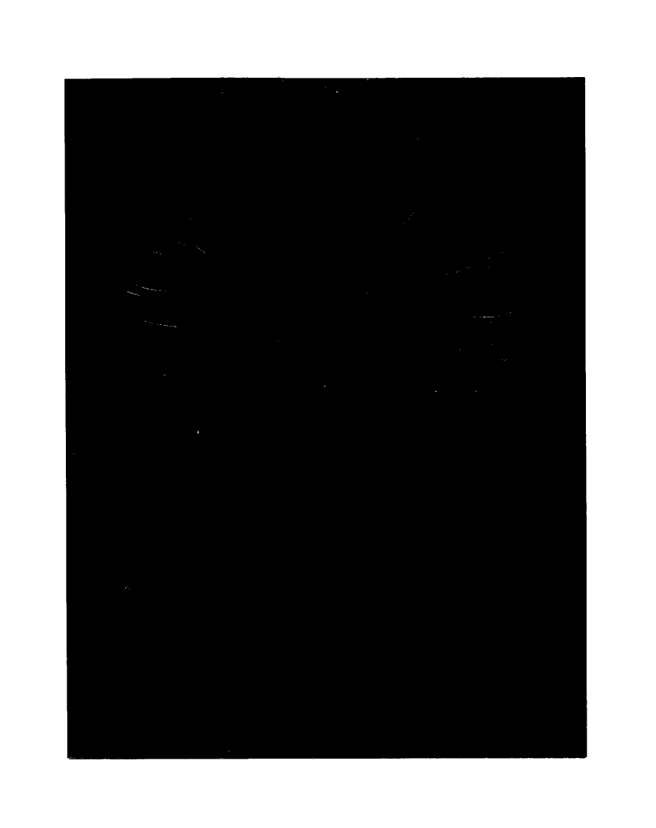

### BACKGROUND OF THE INVENTION

The present invention relates to a new and distinct variety of dahlia plant known by the varietal denomination ‘Diana, Princess of Wales’. The female parent of the new variety is a variety known as ‘Raisers Pride’ and the male parent is unknown.

The new variety of dahlia was bred by the inventor at the request in 1994 of Diana, Princess of Wales, who specified a large-flowered pink form of the class of dahlia called “cactus”. In 1997, the inventor succeeded in breeding a variety with the desired characteristics, using the variety ‘Raiser's Pride’ as female parent. The male parent is unknown. Registration was granted by The Royal Horticultural Society of London as the International Registration Authority for the genus dahlia for the varietal denomination: ‘Diana, Princess of Wales’.

### SUMMARY OF THE INVENTION

The new variety is a dahlia plant useful for growing as a garden plant and floral decoration. Among the traits possessed by the new variety which in combination distinguish it from other varieties are its attractive flowers borne on long stems.

Asexual reproduction by cuttings of the new variety in Stoke Mandeville, Buckinghamshire, England, has demonstrated that the combination of characteristics as described herein for the new variety are firmly fixed and are retained through successive generations of asexual reproduction.

### COMPARISON WITH PARENT

The new variety differs from its female parent in flower color.

### BRIEF DESCRIPTION OF ILLUSTRATIONS

The accompanying color photographic illustration shows a typical specimen of a flower of the new variety in colors as nearly true as possible with an illustration of this type.

### DESCRIPTION OF THE NEW VARIETY

In the following description, color references are to The Royal Horticultural Society Colour Chart, where color values refer to color plate numbers in this chart.

The following observations are of plants described and grown outdoors in Stoke Mandeville, Buckinghamshire, England.

The new variety has not been observed under all possible environmental conditions. The phenotype may vary with variations in environment such as temperature, light intensity, day length and cultural conditions.

Plant:

Form.—Upright, bush.

Height.—About 4 feet.

Branching characteristics.—From point of first pinching about 4 to 7 shoots.

Flower stem.—From point of branching, long, strong.

Length.—Well proportioned, about 30 inches.

Color.—Mainly purplish, near 183B, but with green, near 137B to 144B, linear shading.

Foliage:

Quantity.—Leafy, well balanced.

Size of leaf.—Individual mature leaf about 5 inches long; 3 inches wide at center.

Shape of leaf.—Mature leaf mostly pinnate; oval pointed.

Leaf edge.—Slightly serrated.

Texture.—Smooth, juvenile foliage has a light gloss.

Leaf upper side.—Matte.

Leaf under side.—Matte.

Color:

Leaf base of elongated flower stem.—Base of mature leaf near 147B with tones of near 183B.

Basal leaf of flower stem.—Top of mature leaf near 137A to 137B; underside near 138A to 138B.

Venation.—Non distinctive.

Petiole length.—About 2 inches to 2½ inches.

Stipules.—None.

Bud:

Form.—Round.

Size.—At first sighting, pea size.

Opening rate.—About 4 weeks to mature.

Color of ray florets.—Light coral at 2½ weeks; changing to mature color by 4 weeks.

Flower:

Inflorescence.—Terminal; main bud and two side or wing buds which are normally removed in commercial cultivation.

Type.—Composite.

Diameter.—About 6 inches to 9 inches.

Number of flowers.—Usually 3 per cluster.

Bloom:

Type.—Composite.

Form.—Double cactus flowered.

Borne.—Terminal.

Size.—Variable according to cultivation; generally between about 5 inches and 8 inches wide; between about 3 inches and 5 inches deep.

Shape.—Rounded.

Color tonality from a distance.—Constant, true.

Lasting quality.—About 7 days, average for type.

Color.—Purple/violet.

Inner ray petals.—Short; evenly radiating.

Outer ray petals.—Long mature outer.

Point of petal attachment.—Carpitrulum.

As bloom ages.—Outer petals deteriorate exposing disc florets.

Petal tip.—Near 38B to 38C.

Petal blade.—Near 38B to 38C with lines of near 35C.

Petal base.—Near 6B to 5B with edge near 4A.

Stamen tip.—Near 14C.

Fragrance.—None.

Reproductive organs:

Stamens.—Stamens, anthers, pollen and pistils are typical of the species type; composite.

Character of ovaries.—Singular.

I claim:

1. A new and distinct dahlia plant of the variety substantially as shown and described.
